(19) United States
(12) Patent Application Publication
(10) Pub. No.: US 2026/0331240 A1
(43) Pub. Date: Oct. 22, 2026

(54) System and Method for Central Retrieval and Control of Distributed and Incomplete Records Repositories, and Detection of Trace Hallucinations by Ambient Scribes

(71) Applicant: Aleida Lanza, Miami, FL (US)

(72) Inventor: Aleida Lanza, Miami, FL (US)

(21) Appl. No.: 19/648,460

(22) Filed: Apr. 15, 2026

Related U.S. Application Data
(60) Provisional application No. 63/791,612, filed on Apr. 20, 2025.

(51) Int. Cl. G16H 10/60; G06F 16/215; G06F 21/31

(52) U.S. Cl. CPC G16H 10/60; G06F 16/215; G06F 21/31

(57) ABSTRACT

A server for managing records stored in one or more third-party databases is provided. The server is configured to validate users of server and send commands to third-party databases storing information about the user. The server can format the commands to comply with access rules of the third-party databases and generate composite records combining records from various third-party databases storing records related to the user. The server can also identify breaches of security or rules for managing records stored on third-party databases. The server can be designed to be interoperable with databases storing medical records and related information and to comply with rules related to their storage, including the HITECH Act.

### REFERENCE TO RELATED APPLICATIONS

This patent application claims the benefit under 35 U.S.C. § 119(e) of United States Provisional Patent Application No. 63/791,612, filed Apr. 20, 2025, which application is incorporated herein by reference.

### BACKGROUND OF THE INVENTION

Over the past fifty years, there has been a steady increase in the number and detail of records created and maintained in the United States. These records preserve important and sensitive information that pertains to both individuals and business organizations. Over the last twenty-five years, much of this information has been digitized and is accessible one way or another online. However, in the United States of America, information relating to individuals and business organizations is not centrally stored, even within certain domains or fields of activity. Instead, the information is controlled by variety of entities, both public and private. Often, the information stored in each of these digital repositories is incomplete. Sometimes, information stored in a digital repository is inaccurate, perhaps because it was improperly entered in the first place or later corrupted or is out-of-date and needs to be updated. The records stored in these digital repositories can be duplicative.

Because the information which is commonly stored regarding individuals and some businesses is highly sensitive, either because it is private in nature or because it is capable of being misused, governments at both the state and federal level have introduced laws regulating access and how these records must be handled by the organizations storing them. In addition, many private organizations have implemented their own internal rules for the handling of client, customer, employee, or other third-party records. Often, these laws and regulations grant rights to certain parties, such as the subject of the record, his or her representatives, or trusted professionals, to access, review, delete, and/or update the record. These laws can also require custodians of digital records to report promptly any breaches of their policies or any applicable rule or regulation in the storing or access of the record. The complexity and overlapping nature of these regulations presents technical challenges to these custodians to ensure that the computer systems which store the digital records have the capability to monitor, track, and preserve the records, how they are updated, and by whom they are accessed.

Nowhere are these issues more pressing than in the field of healthcare. The institutional barriers for patients finding, accessing, or sharing personal health information (PHI) are challenging, and may involve layers of duplicative efforts by those parties that either due to their roles or the law need to access this information, including clinical staff, legal teams, claims managers, and patients themselves. Further, the organizations that serve as gatekeepers to this information, such as health information exchanges (HIE), also force patients to first identify the entity hosting their PHI before they can demand a right of access, instead of automatically detecting and providing the patient with a detailed list of all healthcare providers who treated the patient and which controls patient PHI. In addition, the various custodians of digital records store these records in a variety of largely proprietary computer systems, which typically are not interoperable with each other. These barriers to interoperability are technologically constraining because digital health systems often cannot communicate across different platforms, requiring matching software that creates dangerous information gaps and delays in critical care decisions, which drive up costs throughout the healthcare system. Moreover, in 2009, Congress passed into law the Health Information Technology for Economic and Clinical Health (HITECH) Act, which greatly expanded patients’ access to their healthcare information. However, digital health systems and other organizations which store PHI have not kept up with this Act and still have difficulty ensuring that their data systems implement its requirements. While HIPAA was enacted in 1996, the HIPAA Privacy Rule was enacted in 2003 to regulate the transfer of such privileged data between businesses that must be visible to patients.

Despite extraordinary investments by our nation in advancing interoperability in digital health information, fragmentation of digital health record management systems persists, which hinders a variety of stakeholders from efficiently and effectively accessing these digital records: patients, healthcare providers, financial institutions, such as insurance providers, legal professionals, and researchers, such as academic or government researchers. Each of these stakeholders can benefit from access to a complete digital health record and related digital records (such as those recording insurance claims) to effectively support patients, even keeping in mind that certain parties have limited rights of access to third-party PHI (such as researchers who may only access de-identified data with patient consent). Artificial intelligence is now deployed in most healthcare settings for scribe functions that may give rise to hallucinations, which may be inadvertently made part of the patient’s record. These hallucinations may not be visible to patients. Currently, stakeholders must painstakingly gather and analyze the PHI that they need, embedding costly inefficiencies that ultimately burden patients and the healthcare system more generally, with increased delays, higher healthcare costs, higher insurance premiums, and higher litigation expenses, including the editing of hallucinations which are not part of any patient’s course of treatment.

Another issue is the lack of coordination between a patient’s digital health records and those wearable devices and other real-time monitors/sensors that are, for instance, commonly used in a hospital or clinical environment. Because current digital healthcare record management systems ignore wearable devices and health applications, they fail to establish comparative baseline vital statistics for any patient, or offer historic data for comparative studies to current sensor driven health data. In modernizing how HIEs connect digital patient records to wearable devices, health sensors, and health applications running on the patient’s or a third-party’s computing system, healthcare professional can leverage and integrate the digital information collected by these wearable devices, sensors, and health applications to eliminate barriers to patient health, education, engagement, and deliver better, more coordinated care, reduce burdens across environments, and predict the costs for accessing a complete history of a patient’s known medical record.

Other sectors of the United States economy face similar challenges regarding the information collected and stored as part of conducting business or as required by applicable law. One such sector is the financial sector. Banks, insurance companies, lenders, ratings agencies, and others collect a wide variety of information about their customers which are then stored digitally and made accessible internally to those in the organization and others online. Further, due to the sensitive nature of the information collected and/or generated by financial institutions (such as account numbers, social security numbers, full names, addresses, transaction history, etc.), various government agencies, at both the state and federal level, have implemented laws and regulations governing the collection, access, updating, and preservation of financial records. Once again, different custodians of these digital financial records use proprietary computer systems which pose challenges regarding interoperability. Moreover, each financial institution can only store or track electronically that information provided to it or those transactions to which it was a party. As a result, computer systems for storing financial information also tend to contain digital records that are incomplete, potentially inaccurate, and/or out-of-date. The subjects of these digital financial records and other interested parties, such as law enforcement, other financial institutions, and researchers must painstakingly access each individual computer system and create a master digital record, not knowing if that digital record is complete, fully accurate, or contains altered or corrupted information regarding the subject. Further, these computer systems are not capable of comparing newly generated electronic financial data with a complete and accurate record for the relevant subject in real time to provide reliable and timely electronic information that, for example, can be used to validate a digital financial transaction.

Despite numerous technological advances in information technology and billions of dollars in investment in the modernization of computer systems, individuals and business organizations (the subjects of these digital records) in the modern economy are still faced with a patchwork of proprietary systems storing their sensitive digitized information where each record might be incomplete or possibly contain incorrect or old information. Further, these subjects of the digital records lack the capability to discover if their records have been digitally maintained in accordance with applicable laws and regulations or in accordance with the digital document storage policies of the custodian organization. In addition, current electronic platforms for accessing digital records stored on third-party computer systems lack the ability to integrate other sources of digital information related to the subject (such as a personal or wearable device, health sensor, etc.) with the digital records stored on the third-party computer systems, depriving users of these computing systems and the subjects themselves of a complete picture on which to base decisions. Thus, there is a long-felt need for systems and methods to query third-party computing systems storing digital records containing information relating to a subject and access those digital records to create a complete, up-to-date, and accurate master digital record for access by a user. There is a further long-felt need for systems and methods of incorporating digital information generated by synchronous sources such as personal electronic devices (or the applications running on them), wearable devices (such as smartwatches), sensors (such as those sensors for monitoring a patient’s vital signs in a hospital or clinical setting) to provide a user or the subject himself or herself with a real-time unified view drawn from all available electronic data sources. There is also a long-felt need for systems and methods for providing an electronic platform for the user that it is able to query third-party computing systems storing digital records of a subject and determining how and when those digital records have been updated or altered and by whom and whether the digital records stored by the third-party computer system have been electronically stored in accordance with a set up rules defining digital access and transmission, duration of storage, access control, etc. and when and under what circumstances a digital security breach or unauthorized electronic access took place. Further, there is a long-felt need for systems and methods for providing an electronic platform for a user which is able to query third-party computing systems storing digital records of a subject or subjects and create a composite digital record which implements restrictions as to what digitally stored information can be included in the composite digital record based on the access rights of the user and the nature of the subjects.

### BRIEF STATEMENT OF THE INVENTION

The invention broadly relates to the field of online electronic records management and access control.

### SUMMARY OF THE INVENTION

The present invention broadly comprises a computing platform and related methods for aggregating, tracking, sharing, and monitoring the access and control of sensitive records electronically stored in disparate formats in third-party computing systems with online interfaces. The computing platform is accessible to users (including the subjects of the electronic records stored by the third-party computing systems) via a website, an application running on a computer, smartphone, tablet, or other electronic device, or through other proprietary software. The user is capable of verifying his or her identity and/or access rights by inputting identifying information to the computing platform by inputting text via a keyboard or software keyboard, by scanning documents with personal identifying information (such as a driver’s license, passport, social security card, insurance card, etc.), or through biometric input (such as a retina scan, fingerprint or palm scan, etc.). Once the user is validated by the computing platform, the computing platform queries all known repositories for applicable electronic records in the given domain (for example, in finance, banks, credit card companies, mortgage companies, etc. or for medical information, insurance companies, healthcare providers, government databases, etc.). The queries can be executed either through the third-party computer systems’ application programming interfaces (APIs) or via automated email or similar electronic requests. The computing platform receives the electronic records, either through the APIs of the third-party computing systems or through other electronic means (such as through an email or email attachment or by downloading them via file transport protocol (FTP)). The user can also upload electronic documents in his or her possession into the computing platform. The computing platform assembles a complete record of the subject from the information received, eliminating duplicate entries, updating old information from data sources with current information from other data sources, and flagging or eliminating inaccurate or incomplete information. The complete record of the subject is searchable and the user can run queries on it on the computing platform. Further, the computing platform is capable of providing the user with access controls to allow the user to share the compete record of the subject, or a portion thereof, with third-parties including academics and researchers. These access controls further provide the user with the ability to redact, anonymize, or pseudo-anonymize the complete record, or the portions thereof for sharing, prior to sending.

The computing platform monitors and tracks how electronic records have been stored, accessed, and updated on the third-party computer systems to which it has access. The computing platform is capable of recording and storing information regarding whether an electronic record has been updated, by whom, and what was changed. The computing platform is also capable of requesting access histories from the third-party computer platforms to determine if there has been a security breach or a violation of that third-party computer platform’s access rules (whether or not those access rules are derived from an applicable law or regulation). Earlier versions of electronic records can be requested by the computing platform, if they are electronically stored by the third-party computing system.

The computing platform can also access historical data and data created in real-time from electronic devices related to the subject such as applications running on smartphones, data collected by smart watches, other personal devices, and (in the context of medical care) electronic sensors monitoring the subject. The computing platform provides the user with applications and analysis tools to compare the data synchronously collected by the electronic devices with the complete record of the subject to view trends, make comparisons, identify anomalies, or to flag issues.

The present invention further relates to systems and methods, including those implemented using cloud-computing resources, to provide a computing platform for a patient to control, detect, query, extract, transform, load, save, and assert the patient’s right of access for their complete known medical records and other personal health information. The computing platform is further configured to integrate historic vital data with and patient health applications and wearable devices that generate historic vital timelines obtained from a patient’s medical records for comparative analytics of vital record data sources captured by health applications, medical sensors, and/or wearable device (e.g. blood glucose, blood oxygen, blood pressure, heart rate, respiratory rate, heart rate; smart watch, smart device, etc.) to provide the patient with a complete, accurate, up-to-date view of his or her medical history, PHI, and relevant medical readings. Other relevant data relating to the patient’s medical and health history, such as insurance information (health benefits, claim history, etc.) is also accessible via the computing platform. The computing platform can also be used by those professionals granted sufficient access rights by the patient to review the patient’s history to advance the patient’s medical treatment, pay insurance claims or medical bills, or gather information for legal matters. The computing platform allows patients to find, demand access, download, store, and share a complete health insurance claim history from those insurance sources with accessible electronic records databases. The computing platform is capable of implementing access rules with respect to the electronic records that are accessible on third-parties’ computer systems and is further capable of detecting violations of access which occurred on a third-party computer system with respect to a patient’s electronic records. The computing system can implement a patient’s demand for right of access to any healthcare provider, relay an electronic notice to the payor of any non-compliance by the payee, and request an investigation of a claim into the access and storage of a patient’s medical records as required by applicable right of access laws. The computing platform can validate the identity of the patient through the input of patient identifying information, including through scanned or typed insurance information. The computing platform is thereby capable of tracking the patient’s current and past insurance by type, duration and time of coverage, etc. The computing system is interoperable with the online systems of third-parties, including health insurance claim portals, and is configured to search and extract all claim and other related insurance data (such as explanation of benefits data) from all available electronic sources of healthcare coverage. This includes being Health Level 7 (HL7) compliant for exchanging and sharing electronic health information with other compliant systems and for the detection of HL7 error codes related to records stored on third-party computing systems. The computing platform is configured to generate a comprehensive list of all healthcare providers to the patient and a comprehensive history of the patient’s insurance claims and interactions. The computing platform is further capable of detecting and eliminating duplicate provider entries in the patient’s electronic records stored on third-parties’ computing systems, generating a complete and accurate list of the patient’s past and current healthcare providers. The computing system is capable of accessing the patient’s electronic records via the application programming interfaces of third-party computer systems or via query and transmission by fax and/or email.

The computing platform is configured to permit patient control access, and the terms of its access, over his or her comprehensive medical and related information. The computing platform allows a user to grant access to his or her medical records and related information, or a portion thereof, to insurance companies, financial advisors, legal professionals and other trusted professionals. The computing platform allows a user to grant electronic access to his or her medical records and related information, or a portion thereof, in an abridged, anonymized, or pseudo-anonymized format with researchers, government entities, and other organizations who also have access to the computing platform.

The computing platforms further provides the patient with the capability to download his or her full medical record and related information, or a portion thereof, send it, or a portion thereof, to a third party (such as an insurance company), make requests for further information in accordance with rules governing the access and storage of such electronic records (including those based on local, state, and federal laws and regulations), identify breaches in access and storage rules, examine the version histories of his or her medical records, and run queries on his or her full medical record and/or correlate portions of the full medical record with synchronous electronic data provided by a smart watch, personal device (or an application running on it), or medical sensor.

In certain embodiments, a system for managing records stored remotely is provided. The system comprises at least one database and at least one server. Furthermore, the at least one server is configured to perform a method for managing records stored remotely. The at least one server validates the identity of a user by authenticating at least one document or one piece of information related to the user against identity information stored on a third-party database. Additionally, the at least one server receives from a first third-party computer system at least one record stored on the first third-party computer system. The at least one server receives from a second third-party computer system at least one record stored on the second third-party computer system. Additionally, the at least one server combines the at least one record from the first third-party computer system with the at least one record from the second third-party computer system into a combined record and making that combined record accessible on the at least one server.

In certain embodiments, a computer-implemented method for managing records stored remotely is provided. The computer-implemented method validates the identity of a user by authenticating at least one document or one piece of information related to the user against identity information stored on a third-party database by a server. Additionally, the computer-implemented method receives at the server from a first third-party computer system at least one record stored on the third-party computer system. The computer-implemented method receives at the server from a second third-party computer system at least one record stored on the second third-party computer system. Furthermore, the computer-implemented method combines at the server the at least one record from the first third-party computer system with the at least one record from the second third-party computer system into a combined record and makes the combined record accessible on the server.

In certain embodiments, a computer program product comprising a non-transitory computer-readable medium including instructions for execution on one or more computing devices is provided. The instructions of the computer program manage records stored remotely from the one or more computing devices. The instructions of the computer program validate the identity of a user by authenticating at least one document or one piece of information related to the user against identity information stored on a third-party database. Furthermore, the instructions of the computer program receive from a first third-party computer system at least one record stored on the third-party computer system. The instructions of the computer program receive from a second third-party computer system at least one record stored on the second third-party computer system. Additionally, the instructions of the computer program combine the at least one record from the first third-party computer system with the at least one record from the second third-party computer system into a combined record and make the combined record accessible on the server.

It is a general object of the present invention to provide a mechanism through which patients and their providers can ascertain a true validated existing baseline that detects and reports the highest and lowest values for each vital record to assist in contextualizing the real time wearable device data, and flag instances which alter these values to the individual using them.

It is another general object of the present invention to inform the patient and provider of comparative longitudinal vital sensor data based upon the whole existing claim record history and aggregated vital statistics across environments that are not currently available to medicine today.

### OBJECT OF THE INVENTION

It is a general object of the present invention to provide systems and methods and centralized access and control of records maintained on various servers and to generate a master record therefrom.

These and other objects and advantages of the present invention will be readily appreciable from the following description of preferred embodiments of the invention and from the accompanying drawings and claims.

### BRIEF DESCRIPTION OF FIGURES

The nature and mode of operation of the present invention will now be more fully described in the following detailed description of the invention taken with the accompanying drawing figures, in which:

FIG. 1 is a diagram of an exemplary system in accordance with certain embodiments.

FIG. 2 shows a flow diagram of an exemplary embodiment for generating a unified patient record.

FIG. 3 shows a schematic view of an exemplary graphical user interface for interacting with the electronic patient record management system.

FIG. 4 shows a flow diagram of an exemplary embodiment for generating a report of the access history and revision history for a patient’s records.

FIG. 5 is a diagram of an exemplary system in accordance with certain embodiments.

### DETAILED DESCRIPTION

At the outset, it should be appreciated that like drawing numbers on different drawing views identify identical, or functionally similar, structural elements of the invention. While the present invention is described with respect to what is presently considered to be the preferred aspects, it is to be understood that the invention as claimed is not limited to the disclosed aspects.

Furthermore, it is understood that this invention is not limited to the particular methodology, materials and modifications described and as such may, of course, vary. It is also understood that the terminology used herein is for the purpose of describing particular aspects only, and is not intended to limit the scope of the present invention, which is limited only by the appended claims.

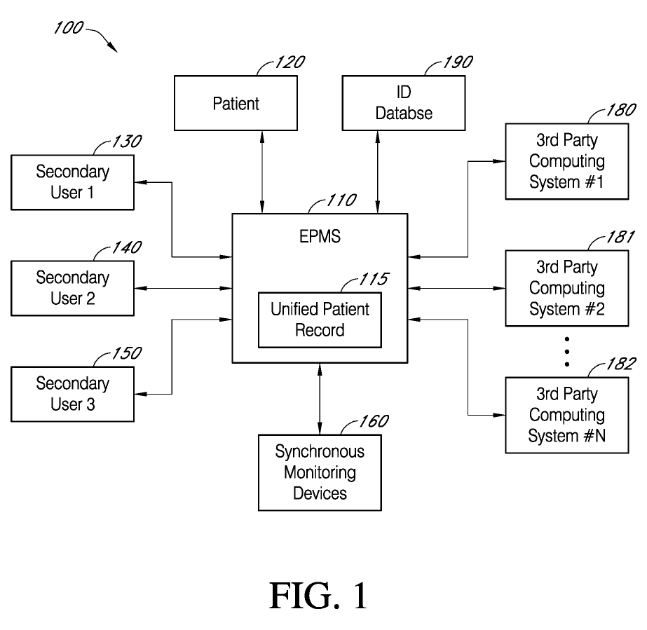

FIG. 1 illustrates elements of an exemplary system 100 for electronic patient record management system (EPMS) 110 for managing access to patient medical records and for patient 120 to generate electronic demands to third-party computing systems 180, 181, …, 182 storing relevant electronic records related to the patient (such as those operated by insurance companies, HIEs, healthcare providers, etc.). The EPMS 110 can send requests to third-party computing systems 180, 181, …, 182 holding relevant records to patient 120 to create a unified patient record 115 containing all available information stored by third-party computer systems 180, 181, …, 182. The requests can be formatted by the EPMS 110 to be compatible with the requirements of the third-party computer systems 180, 181, …, 182, including those requirements deriving from applicable law (such as the Health Information for Economic and Clinical Health Act, or HITECH Act, or similar state, local, or federal laws and regulations) and/or including information necessary for verifying access rights or for authentication (such as names, identification numbers, user identification, passwords, etc.). In certain embodiments, the EPMS 110 provides patient 120 with the means to input electronic records relating to the patient’s 120 medical history including, but not limited to, his or her medical records, insurance records, etc. The EPMS 110 can include optical character recognition (OCR) technology and can parse those records provided by the patient 120 directly or already accessed from the third-party computer systems 180, 181, …, 182 to generate follow-up queries based on omissions or gaps identified in the unified patient record 115. The EPMS 110 can be configured to be able to electronically send queries/requests and receive data, either through their APIs or by another means (such as via email or file transfer protocol or via facsimile), from third-party computing systems 180, 181, …, 182 which store electronic records. EPMS 110 can also send queries and receive data from Employee Retirement Income Security Act (ERISA) records, Medicare records, Medicaid records, supplemental insurance records, private insurer records, healthcare provider records, etc. The EPMS 110 can be configured to periodically query third-party computing systems 180, 181, …, 182 for new information to update unified patient record 115 or the query for new information can be initiated by patient 120 or secondary user 130, 140, and/or 150. In certain embodiments, communications between the EPMS 110 and the third-party computing systems 180, 181, …, 182 are encrypted. Other communications to and from EPMS 110 can also be encrypted.

EPMS 110 can also communicate with one or more synchronous monitoring devices 160 associated with patient 120. Synchronous monitoring devices 160 can include a smart watch, such as an Apple Watch®, a ring sensor, such as an Oura Ring®, or a medical sensor, such as a patient-worn ECG/PPG sensor to monitor heart rate and oxygen levels. Synchronous monitoring devices 160 can send the data that they collect in real time or send stored data periodically. EPMS 110 can store the data received from the synchronous monitoring devices 160 and/or make that information available in real time to patient 120 or to secondary users 130, 140, and 150.

Secondary users 130, 140, and 150 are individuals or organizations with limited access rights to the EPMS 110 and the unified patient records 115 and related records because they are not the subjects of the unified patient records 115 and related records. Secondary users 130 can be researchers and/or research organizations (including academic, government, non-government organizations, or private party researchers). Secondary users 140 can be medical professionals or medical organizations/healthcare providers including, but not limited to, doctors, nurses, hospitals, clinics, and specialists treating patient 120 or otherwise responsible for his or her medical care. Secondary users 150 can be trusted professionals or professional business organizations including but not limited to attorneys, paralegals, legal secretaries, expert witnesses, law firms, or investigators. In certain embodiments, EPMS 110, is capable of altering the unified patient record 115 and related documents by redacting identifying information (such as name, social security number, date of birth, address, insurance information, payment information, etc.) before sending them to secondary users 130, 140, and 150. EPMS 110 can also be configured to aggregate, anonymize, and/or pseudo anonymize patient records 115 and related documents before making those documents accessible to secondary users 130, 140, and 150.

EPMS 110 is also capable of exchanging information with ID database 190. ID database 190 stores information about patient 120 and secondary users 130, 140, and 150 that can be used to authenticate them on EPMS 110 and/or third-party computing systems 180, 181, …, 182. ID database 190 can be a database maintained by a local, state, or federal agency (such as a state department of motor vehicles or the social security administration) or it could be maintained by a private entity (such as an insurance company or healthcare system). EPMS 110 can have access to two or more ID databases 190, for example databases maintained by different agencies or organizations. In certain embodiments, EPMS 110 maintains its own ID database 190 for at least some secondary users 130, 140, and 150 and/or patients 120 and/or ID database 190 forms part of one or more third-party computing systems 180, 181, …, 182.

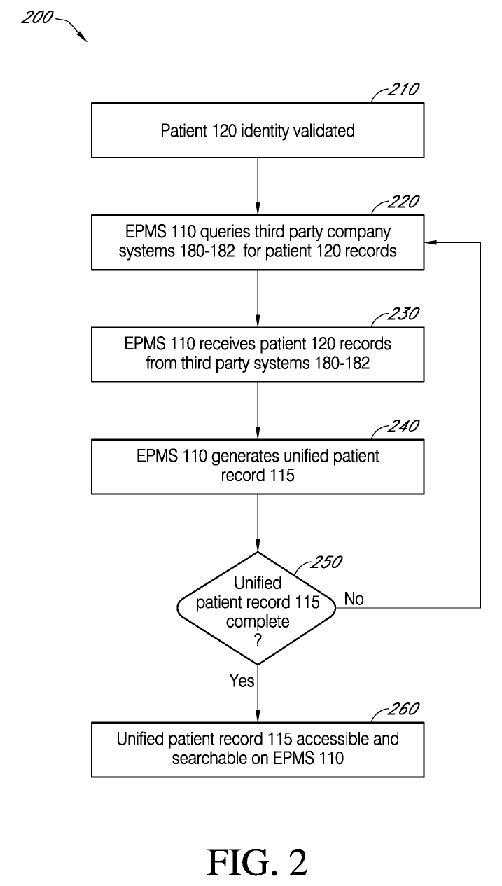

FIG. 2 is a block diagram that illustrates an exemplary process flow 200 for creating a unified patient record 115. At step 210, EPMS 110 validates the identity of patient 120. EPMS 110 can validate the identity of patient 120 by authenticating documents or information inputted or uploaded to EPMS 110. This information can include the patient’s 120 name, address, social security number, age, insurance information, telephone number, among other things. Documentation can include a driver’s license, state identification card, passport, birth certificate, social security card, military identification card, insurance card or others. Documents can be scanned in at the personal electronic device of patient 120. Information can be inputted on the personal electronic device of patient 120 (by typing, for example, such as through a soft or traditional keyboard). In certain embodiments of the invention, additional documents, such as insurance policies, medical records, etc., can be uploaded to the EPMS 110 from the personal electronic device of patient 120 at step 210. At step 220, EPMS 110 queries third-party computing systems 180, 181,…, 182 for records pertaining to patient 120. In certain embodiments, prior to step 220, the third-party computer systems 180, 181, …, 182 searched have been pre-enrolled, providing EPMS 110 with special access, including prioritized searching, more access rights to data resources, etc. In certain embodiments, the patient 120 can initiate a query by a simple request such as selecting a button on a graphical user interface displayed on his or her personal electronic device. The structure of the query at step 220 can be formatted to comply with APIs of third-party computing systems 180, 181, …, 182. In certain embodiments, EPMS 110 is interoperable with computer systems for storing medical or related records using Fast Healthcare Interoperability resources (HL7/FHIR), Medicare Bluebutton API, Medicaid API, MAO API, and other APIs used by private insurers, HIEs, and healthcare providers. The structure of the query at step 220 can also comply with any laws or regulations (including federal laws such as HITECH, state laws such as the California Consumer Privacy Act, or CCPA, or internal organizational rules) and/or provide information for authentication or to receive access rights. At step 230, EPMS 110 receives electronic records from third-party computing systems 180, 181, …, 182 relating to patient 120. The records received by EPMS 110 are parsed (as well as any records uploaded from the personal electronic device of patient 120) to identify references to missing documents or records. Those records not provided in text format can be converted to searchable text format using OCR technology. These references can include information regarding insurance companies, government insurance programs (such as Medicare, Medicaid, Veterans Affairs), hospital stays, medical procedures (such as surgeries, prescriptions, or other treatments), healthcare providers (such as doctors, nurses, healthcare systems), etc. At step 240, EPMS 110 generates the unified patient record 115. In creating the unified patient record 115, EPMS 110 can remove duplicate entries in the unified patient record 115, update outdated or inaccurate information held in some records with current information received in other records from third-party computing systems 180, 181, …, 182, of from patient 120, index information in the unified patient record 115 with the original records retrieved from third-party computing systems 180, 181, …, 182 (allowing retrieval of the original record later, if necessary), structure the unified patient record 115 to be searchable, etc. At step 250, EPMS 110 determines if unified patient record 115 is complete, for example, in part by using the analysis of the source records performed in step 230. If the unified patient record 115 is not complete, EPMS 110 returns to step 220 to query third-party computing systems 180, 181, …, 182 again for the missing information. EPMS 110 can update the query to target only certain electronic records or target only certain third-party computing systems 180, 181, …, 182. In certain embodiments, EPMS 110 keeps track of how many times it has queried third-party computing systems 180, 181, …, 182. After a certain number of times, or after a certain level of completeness in the unified patient record 115 has been reached, EPMS 110 can proceed to step 260. At step 250, if EPMS 110 determines that unified patient record 115 is complete, EPMS 110 proceeds to step 260. At step 260, the unified patient record 115 is now accessible on EPMS 110.

Patient 120 can view unified patient record 115 on a personal electronic device. Unified patient record 115 is also ready to be accessed or sent to secondary users 130, 140 and 150. Unified patient record 115 can be searchable, allowing patient 120 and secondary users 130, 140, and 150 to query the unified patient record 115. In certain embodiments, EPMS 110 can include an artificial intelligence (AI) agent to provide analysis of the unified patient record 115, respond to questions, and/or to summarize all or portions of the unified patient record 115. In certain embodiments, patient 120 can send additional requests for records to third-party computing systems 180, 181, …, 182. These requests for additional records can comply with the rules and regulations governing medical records and their request and retrieval, such as Health Insurance Portability and Accountability Act (HIPAA) or the HITECH Act.

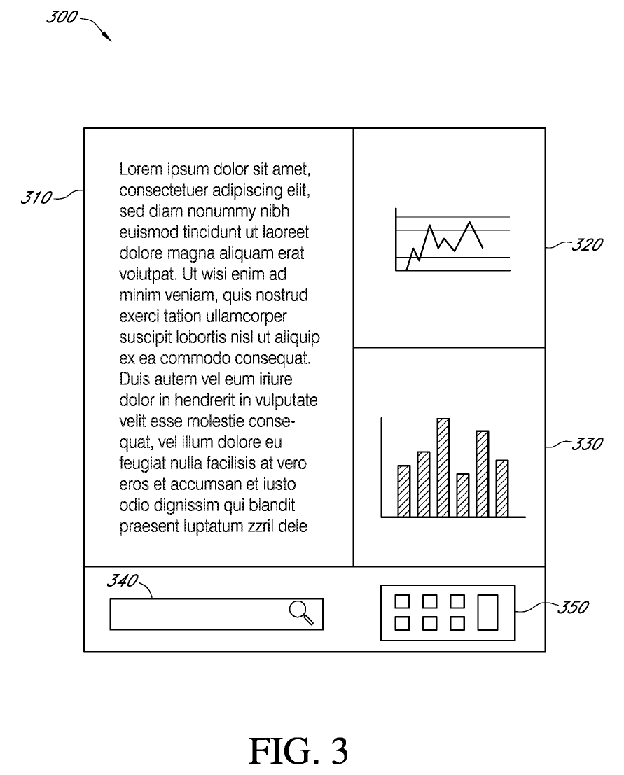

FIG. 3 is an illustration of graphical user interface (GUI) 300 in accordance with certain embodiments. GUI 300 can be displayed on the personal electronic device of patient 120 and on the personal electronic devices of secondary users 130, 140 and 150. GUI 300 can include record viewer 310, sensor viewer 320, chart viewer 330, search prompt 340, and control panel 350. In certain embodiments, GUI 300 is modified so that certain functionality is disabled or certain information cannot be displayed based on whether GUI 300 is being displayed on the personal electronic device of patient 120 or on the personal electronic device of secondary user 130, 140 or 150. Record viewer 310 provides a view of unified patient record 115. The unified patient record 115 can be scrolled within record viewer 310. Text and graphics within the record viewer 310 can be enlarged or made smaller and text and various portions of the unified patient record can be hyperlinked to other portions of the unified patient record or to the original record (or records) stored on the third-party computing systems 180, 181, …, 182. The original records can also be viewed within record viewer 310. Sensor viewer 320 can present data received from one or more synchronous monitoring device(s) 160. Sensor viewer 320 can present data in real time or it can present data stored over a given period of time in the past. The x-axis and the y-axis of sensor viewer 320 can present any units of measurement at any resolution. In certain embodiments, the x-axis shows time in units of measurement including seconds, minutes, hours, or days. Sensor viewer 320 allows for zooming in and zooming out. Search prompt 340 can be used to enter search terms. In certain embodiments, search prompt 340 can be used to enter text searches. Search prompt 340 can also include an AI agent to facilitate searches of the unified patient record 115 and/or provide summaries of patient data in the record viewer 310. Control panel 350 provides means for controlling EPMS 110 and the unified patient record 115. Control panel 350 includes an interface (such as a request button) for requesting records from third-party computing systems 180, 181, …, 182. Control panel 350 includes controls for controlling and selecting sensor output from synchronous monitoring devices 160, including whether to present real time data or historical data. Control panel 350 can also be used to create comparisons between inputs from multiple sources (such as the synchronous monitoring devices 160 and/or unified patient record 115) in chart viewer 330.

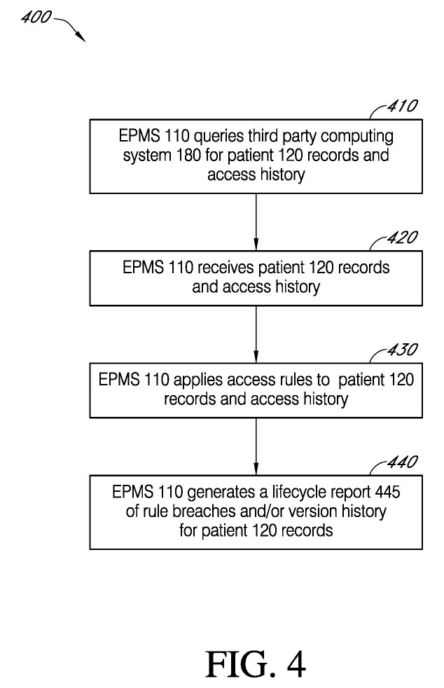

FIG. 4 is a block diagram that illustrates an exemplary process flow 400 for generating an access and revision report for those records incorporated into unified patient record 115. At step 410, EPMS 110 queries third-party computing system 180 for the records of patient 120 and their access history. At step 420, EPMS 110 receives the records of patient 120 and their access/revision history. In certain embodiments, the access or revision history of the record can, at least in part, be stored in the metadata of the record. Access and revision history can also be stored in a log maintained by the database or operating system storing the record or in a different file separate from the record. At step 430, EPMS 110 applies access rules to records of patient 120 to determine if there are any breaches of the rules. In certain embodiments, the access rules applied can be based on a federal law such as HIPAA or HITECH or on a state law such as the California CCPA or from another source of law. These access rules can be maintained on EPMS 110 and updated periodically based on changes in underlying law or based on the requirements of patient 120 or some third party (such as an insurance company or legal advisor). In certain embodiments, only a subset of the rules or only revision history (for example based off of a request by patient 120 or another party) is collected for each record or a subset of the records. At step 440, EPMS 110 generates a lifecycle report 445 of the rule breaches and/or version history for the records of patient 120. This lifecycle report 445 can then be made available to patient 120 and secondary users 130, 140, and 150 through their personal electronic devices. The process 400 can be repeated for other third-party computing resources 181, …, 182. The lifecycle report 445 can also be searchable and/or include hyperlinks to the records breached or updated and to earlier versions of updated records, if they exist. In certain embodiments, the lifecycle report can include information about when doctors’ and other medical staffs’ notes were generated by an artificial intelligence agent, whether those sections were revised or not, how, and/or by whom. In certain embodiments, EPMS 110 can scan a claim record history of patient 120. EPMS 110 can identify hallucinations made by an ambient AI scribe application when generating its summary when the generated summary is edited by a health provider prior to saving the summary as part of the health record of patient 120. In instances where the original record still exists on third-party computing systems 180, 181, …, 182, EPMS 110 can detect how many edits were made and retrieve the voice print for comparison (e.g. trace editing pre-acceptance) and compare to health record of patient 120 as accepted by the health provider. In certain embodiments, EPMS 110 can request data and capture it from third-party computing systems 180, 181, …, 182 every thirty days. EPMS 110 can also request that all or a portion of the data related to the health records of patient 120 are preserved and not erased, which maintains the records for further use by EPMS 110 or secondary user 130, 140, or 150 or patient 120.

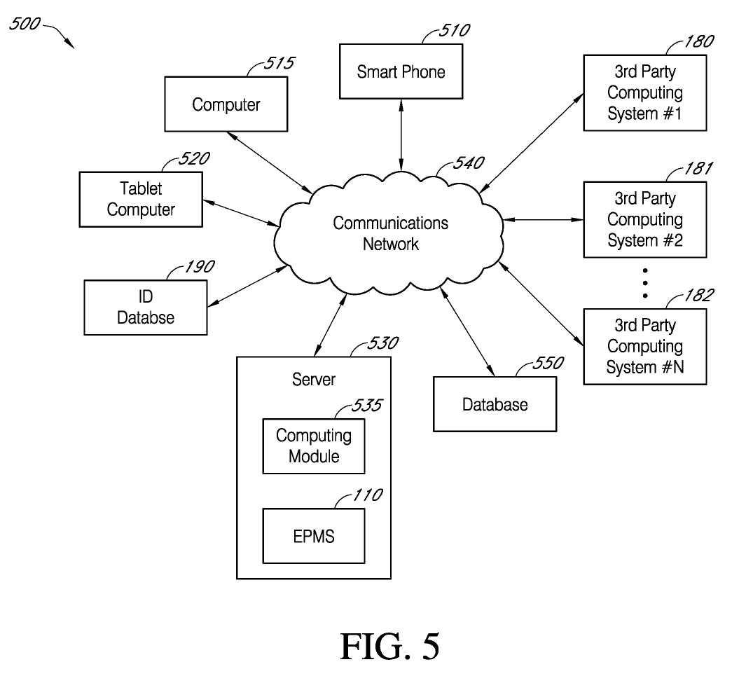

FIG. 5 illustrates an exemplary computer network 500 or similar digital processing environment for implementing EPMS 110, in accordance with some embodiments. Personal electronic devices include smart phone 510, computer 515 (which can be a laptop or desktop device), or table computer 520 which can connect with server 530 via communications network 540. Personal electronic devices (smart phone 510, personal computer 515, and table computer 520) can also be linked through communications network 540 including to each other, other personal electronic devices, other servers, or other electronic services. Personal electronic devices (smart phone 510, personal computer 515, and tablet computer 520) include at least one means of input (such as a keyboard, mouse, trackpad, microphone -for voice commands, touchscreen, trackpad, trackball, buttons, scanner, or a combination thereof) and at least one means of output (such as a projector, monitor, touch screen, flat screen, speakers – for audio responses, printer, or a combination thereof). Personal electronic devices (smart phone 510, personal computer 515, and table computer 520) can also output information and accept commands through another device wirelessly connected or hardwired to it, such as a smartwatch, smart television, or smart speaker (not shown). The communications network 540 can be a remote access network, a global network (e.g., the Internet), a worldwide collection of computers, local area or wide area networks, and gateways that currently use standard protocols (TCP/IP, Bluetooth®, Near-Field Communication (NFC), etc.), or a combination thereof. Any communication architecture, either wired, wireless, or a combination thereof, capable of facilitating the exchange of information between at least one of smart phone 510, personal computer 515, or tablet computer 520 and server 530 can be used to implement communications network 540. Server 530 can access data, including unified patient records 115 and lifecycle reports 445, stored on database 550. Server 530 is also connected to third-party computing systems 180, 181, …, 182 via the communications network 540.

Server 530 is capable of executing all of the processes and accessing all of the data necessary to host EPMS 110. Server computer 530 includes at least one computing module 535. The one or more computing modules 535 can be configured to execute any computer program instructions that are stored or included on the firmware, in memory, and/or on the hard drives of server computer 530 and can include one or more central processing units (CPUs), one or more microprocessors, one or more microcontrollers, or one or more controllers. The one or more computing modules 535 can be a one or more complex instruction set computing (CISC) microprocessors, one or more reduced instruction set computing (RISC) microprocessors, one or more very long instruction word (VLIW) microprocessors, one or more graphics processor units (GPU), or one or more application specific integrated circuits (ASICs). The one or more computing modules 535 can also include one or more digital signal processors, or any other type of processor or processing circuit capable of performing desired functions, or any subset thereof. Server 530 also includes network controllers for communicating with communications network 540. Server 530 can also include firmware, memory, or one or more hard drives for storing the instructions and data for hosting the EPMS 110.

In certain embodiments, server 530 can be two or more physical servers which communicate with each other over the communications network 540 or through another communications system. Server 530 can also be run as a virtual machine hosted on another computer system. Server 530 can also be hosted on a cloud computing platform, such as Amazon Web Services® or Microsoft Azure®.

Database 550 can be a relational database capable of handling queries and updating itself using the structured query language. In certain embodiments, database 550 runs the Oracle® database, Microsoft® SQL server, or an open source database software system, such as MySQL®. In certain embodiments, database 550 is implemented on one or more computing devices using blockchain technology, such as Hyperledger®. Database 550 can run one more computer systems that include one or more processors, memory, one or more storage devices (such as hard drives), and a network controller to communicate with communications network 540. Database 550 is capable of receiving queries from the server 530 and returning the data requested by the query, including returning unified patient records 115 or lifecycle reports 445. Database 550 is capable of storing unified patient records 115, lifecycle reports 445, and copies of records (including early copies of these records or portions thereof). Database 550 is capable of preserving the relationships between the unified patient records 115, lifecycle reports 445, and the source records used to generate them originally stored on third-party computing systems 180, 181, …, 182.

In certain embodiments, the same hardware or cloud computing resources can be used to host both the server 530 and the database 550. In certain embodiments, aspects of the EPMS 110 (such as its various features, functionalities, or processes) can be implemented by the personal electronic devices (smart phone 510, personal computer 515, and table computer 520).

In certain embodiments, commands and data required to implement the EPMS 110 are a computer program product, including a computer-readable medium (e.g., a removable storage medium such as one or more DVD-ROM's, CD-ROM's, diskettes, tapes, etc.). The computer program product can be installed by any suitable software installation procedure on server 530. In certain embodiments, at least a portion of the software instructions can also be downloaded over communications network 540 or some other network capable of transmitting data.

Embodiments or aspects thereof may be implemented in the form of hardware (including but not limited to hardware circuitry), firmware, or software. If implemented in software, the software may be stored on any non-transient computer readable medium that is configured to enable a processor to load the software or subsets of instructions thereof. Computing module 535 then executes the instructions and is configured to operate or cause an apparatus to operate in a manner as described herein.

Further, hardware, firmware, software, routines, or instructions may be described herein as performing certain actions and/or functions of the data processors. However, it should be appreciated that such descriptions contained herein are merely for convenience and that such actions in fact result from computing devices, processors, controllers, or other devices executing the firmware, software, routines, instructions, etc.

It should be understood that the flow diagrams, block diagrams, and network diagrams may include more or fewer elements, be arranged differently, or be represented differently. But it further should be understood that certain implementations may dictate the block and network diagrams and the number of block and network diagrams illustrating the execution of the embodiments be implemented in a particular way.

Accordingly, further embodiments may also be implemented in a variety of computer architectures, physical, virtual, cloud computers, and/or some combination thereof, and, thus, the data processors described herein are intended for purposes of illustration only and not as a limitation of the embodiments.

While example embodiments have been particularly shown and described, it will be understood by those skilled in the art that various changes in form and details may be made therein without departing from the scope of the embodiments encompassed by the appended claims.

Thus, it is seen that the objects of the present invention are efficiently obtained, although modifications and changes to the invention should be readily apparent to those having ordinary skill in the art, which modifications are intended to be within the spirit and scope of the invention as claimed. It also is understood that the foregoing description is illustrative of the present invention and should not be considered as limiting. Therefore, other embodiments of the present invention are possible without departing from the spirit and scope of the present invention.

Various substantially and specifically practical and useful exemplary embodiments of the claimed subject matter, are described herein, textually and/or graphically, including the best mode, if any, known to the inventors for carrying out the claimed subject matter. Variations (e.g., modifications and/or enhancements) of one or more embodiments described herein might become apparent to those of ordinary skill in the art upon reading this application. The inventors expect skilled artisans to employ such variations as appropriate, and the inventors intend for the claimed subject matter to be practiced other than as specifically described herein. Accordingly, as permitted by law, the claimed subject matter includes and covers all equivalents of the claimed subject matter and all improvements to the claimed subject matter. Moreover, every combination of the above described elements, activities, and all possible variations thereof are encompassed by the claimed subject matter unless otherwise clearly indicated herein, clearly and specifically disclaimed, or otherwise clearly contradicted by context.

The use of any and all examples, or exemplary language (e.g., “for example” or “such as”) provided herein, is intended merely to better illuminate one or more embodiments and does not pose a limitation on the scope of any claimed subject matter unless otherwise stated. No language in the specification should be construed as indicating any non-claimed subject matter as essential to the practice of the claimed subject matter.

Thus, regardless of the content of any portion (e.g., title, field, background, summary, description, abstract, drawing figure, etc.) of this application, unless clearly specified to the contrary, such as via explicit definition, assertion, or argument, or clearly contradicted by context, with respect to any claim, whether of this application and/or any claim of any application claiming priority hereto, and whether originally presented or otherwise: there is no requirement for the inclusion of any particular described or illustrated characteristic, function, activity, or element, any particular sequence of activities, or any particular interrelationship of elements; no characteristic, function, activity, or element is “essential”; any elements can be integrated, segregated, and/or duplicated; any activity can be repeated, any activity can be performed by multiple entities, and/or any activity can be performed in multiple jurisdictions; and any activity or element can be specifically excluded, the sequence of activities can vary, and/or the interrelationship of elements can vary. Accordingly, every portion (e.g., title, field, background, summary, description, abstract, drawing figure, etc.) of this application, other than the claims themselves, is to be regarded as illustrative in nature, and not as restrictive, and the scope of subject matter protected by any patent that issues based on this application is defined only by the claims of that patent. The use of the terms “a”, “an”, “said”, “the”, and/or similar referents in the context of describing various embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. In this application “or” and “and/or” are used interchangeably.

1. A system for managing records stored remotely comprising:
at least one database; and
at least one server configured to perform a method for managing records stored remotely, the method comprising:
validating the identity of a user by authenticating at least one document or one piece of information related to the user against identity information stored on a third-party database;
receiving from a first third-party computer system at least one record stored on the third-party computer system;
receiving from a second third-party computer system at least one record stored on the second third-party computer system;
combining the at least one record from the first third-party computer system with the at least one record from the second third-party computer system into a combined record; and
making the combined record accessible on the at least one server.

2. The system of claim 1, wherein the at least one record from the first third-party computer system is a medical record related to the user.

3. The system of claim 1, wherein the at least one record from the second third-party computer system is an insurance record related to the user.

4. The system of claim 1 wherein the combined record is stored on the database.

5. The system of claim 1 wherein the combined record is accessible to the user on a personal electronic device connected to the server via a network.

6. The system of claim 1 wherein the method for managing records stored remotely further comprises eliminating duplicate entries between the at least one record from the first third-party computer system and the at least one record from the second third-party computer system.

7. The system of claim 1 wherein the method for managing records stored remotely further comprises updating an entry of at least one record from the first third-party computer system with information from an entry in at least one record from the second third-party computer system.

8. A computer-implemented method for managing records stored remotely, the computer-implemented method comprising:
validating the identity of a user by authenticating at least one document or one piece of information related to the user against identity information stored on a third-party database by a server;
receiving at the server from a first third-party computer system at least one record stored on the third-party computer system;
receiving at the server from a second third-party computer system at least one record stored on the second third-party computer system;
combining at the server the at least one record from the first third-party computer system with the at least one record from the second third-party computer system into a combined record; and
making the combined record accessible on the server.

9. The computer-implemented method of claim 8, wherein the at least one record from the first third-party computer system is a medical record related to the user.

10. The computer-implemented method of claim 8, wherein the at least one record from the second third-party computer system is an insurance record related to the user.

11. The computer-implemented method of claim 8, wherein the combined record is stored on the database.

12. The computer-implemented method of claim 8, wherein the combined record is accessible to the user on a personal electronic device connected to the server via a network.

13. The computer-implemented method of claim 8 further comprising eliminating duplicate entries between the at least one record from the first third-party computer system and the at least one record from the second third-party computer system.

14. The computer-implemented method of claim 8 further comprising updating an entry from at least one record from the first third-party computer system with information from an entry in at least one record from the second third-party computer system.

15. A computer program product, the computer program product comprising a non-transitory computer-readable medium including instructions for causing one or more computing devices to:
Manage records stored remotely from the one or more computing devices:
validate the identity of a user by authenticating at least one document or one piece of information related to the user against identity information stored on a third-party database;
receive from a first third-party computer system at least one record stored on the third-party computer system;
receive from a second third-party computer system at least one record stored on the second third-party computer system;
combine the at least one record from the first third-party computer system with the at least one record from the second third-party computer system into a combined record; and
make the combined record accessible on the server.

16. The computer program product as claimed in claim 15, wherein the at least one record from the first third-party computer system is a medical record related to the user.

17. The computer program product as claimed in claim 15, wherein the at least one record from the second third-party computer system is an insurance record related to the user.

18. The computer program product as claimed in claim 15, wherein the combined record is stored on the database.

19. The computer program product as claimed in claim 15, wherein the combined record is accessible to the user on a personal electronic device connected to the server via a network.

20. The computer program product as claimed in claim 15, wherein the one or more computing devices further send a request for a record to the first third-party computer system for a record wherein said request is at least formatted or includes information to comply with a rule for accessing the record on the first third-party computer system.
